(19) United States
(12) Plant Patent Application Publication
(10) Pub. No.: US 2013/0263333 P1
(43) Pub. Date: Oct. 3, 2013

(54) Rosa hybrida named 'AUSchris'

(50) ;

(71) Applicant: David C.H. Austin, Wolverhampton (GB)

(72) Inventor: David C.H. Austin, Wolverhampton (GB)

(21) Appl. No.: 13/506,557

(22) Filed: Apr. 27, 2012

(30) Foreign Application Priority Data
Apr. 3, 2012 (EP) CPVO 2012/0765

(51) Int. Cl. A01H 5/00

(52) U.S. Cl. PLT/137

(57) ABSTRACT

A variety of cut rose plant named ‘AUSchris’. The ‘AUSchris’ rose is a plant having beautiful full-petalled, cup-shaped blooms, having at the center of each bloom a button eye of small inward folding petals, an opening color that is a soft creamy apricot, which quickly fades to cream and then to pure white, and a fragrance that is pure myrrh and of medium strength. The petals of the flower drop very cleanly and it forms a most attractive, bushy, but relatively upright that is extremely healthy.

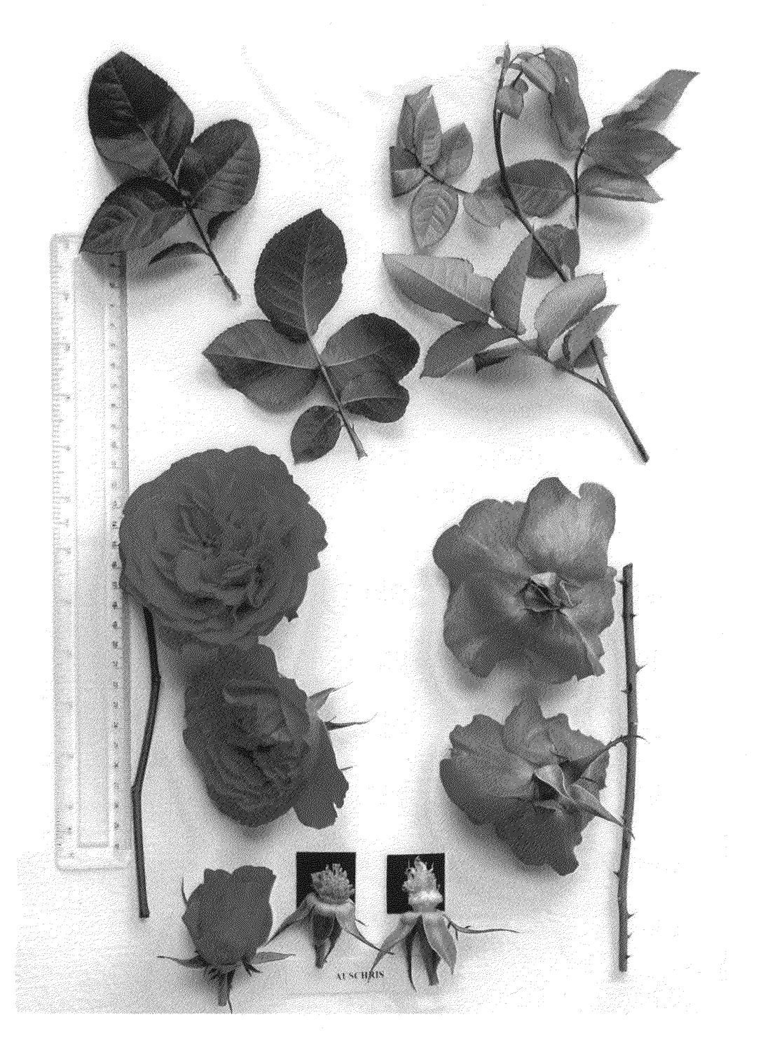

### CROSS-REFERENCE TO RELATED APPLICATIONS

The current application claims priority to a European Community Plant Variety Application No. 2012/0765, filed Apr. 3, 2012, the disclosure of which is incorporated herein by reference.

### CLASSIFICATION

The present invention relates to a new Rosa hybrida.

### VARIETY DENOMINATION

The new plant has the varietal denomination ‘AUSchris.’

### ORIGIN OF INVENTION

This invention relates to a new and distinct variety of rose plant named ‘AUSchris’, which was originated from the cross-pollination of the two ‘unnamed’ seedlings of Rosa hybrida.

### BACKGROUND OF THE INVENTION

The primary objective of this breeding was to produce a variety of luxury cut rose with an unusually long vase life, long elegant magenta pink buds with petals that deepen as they age taking on hints of purple, and a balanced pure rose fragrance with hints of raspberry, red currant, geranium and bergamot that remain through all stages of the flowering.

### SUMMARY OF THE INVENTION

Among the features which distinguish the new variety from others presently available and commercial rose cultivars known to the inventor is the following combination of characteristics: a magenta pink color that fades into a deeper almost purple color with age, a fragrance that remains throughout all stages of life, and a pure rose fragrance with hints of raspberry, red currant, geranium and bergamot.

Asexual reproduction of the new variety by budding as performed outdoors at Bowling Green Lane in Albrighton, Britain shows that the foregoing and other distinguishing characteristics come true to form and are established and transmitted through succeeding asexual propagations. ‘AUSchris’ may be asexually propagated by budding. The budding successfully occurred on the plant/rootstock Rosa cv. ‘Inermis’.

### COMPARISON WITH PARENTS

The seed and pollen parents of ‘AUSchris’ are two unnamed seedlings. The new rose may be distinguished from its seed parent, by the following combination of characteristics: the new variety is deep pink fading to purple with fewer petals and slightly larger flowers, whereas the seed parent is dark red with a smaller bloom and more petals. The new rose may be distinguished from its pollen parent, by the following combination of characteristics: the pollen parent is white, with a hybrid tea form.

### COMPARISON WITH THE CLOSEST COMMERCIALLY AVAILABLE CULTIVAR

The closest commercially available cultivars to the new variety are the ‘AUSglade’ (U.S. Plant Pat. No. 19,161). The new rose may be distinguished from the ‘AUSglade variety by the following combination of characteristics: the ‘AUSchris’ has 65 petals whereas ‘AUSglade’ has 140-150; ‘AUSchris’ has a shallower cup, which is rosette shaped, whereas ‘AUSglade’ has a deep cupped and quartered bloom form; and ‘AUSchris’ has no prickles, whereas ‘AUSglade’ has prickles.

### BRIEF DESCRIPTION OF ILLUSTRATION

The accompanying photograph illustrates the new variety and shows the flowering thereof from bud to full bloom depicted in color as nearly correct as it is possible to make in a color illustration of the character. Throughout this specification, color references and/or values are based upon The Colour Chart of The Royal Horticultural Society (2001) except where common terms of color definition are employed.

### DESCRIPTION OF THE NEW VARIETY

Observations of budded ‘AUSchris’ specimens were made at the nursery at David Austin Roses Limited in Albrighton, Wolverhampton, United Kingdom. The following description is of 1 year-old rose plants of the new variety grown indoors in a greenhouse in Albrighton, Britain in the month of October. Phenotypic expression may vary with environmental, cultural and climatic conditions, as well as differences in conditions of light and soil.
Flower characteristics:
   Blooming habit.—Continuous. The number of blooms per plant during the growing season is profuse, but there are too many to count.
   Bud.—Size: About 4 cm long and 3 cm in diameter when the petals start to unfurl. Form: The bud form is long and pointed ovoid. Color: When sepals first divide, the bud color is red RHS N66A. When half blown, the upper sides of the petals are RHS 67A and the lower sides of the petals are RHS 67A. Sepals: Arrangement: Regular Color: Upper Surface: Green RHS 138A. Lower Surface: green RHS 138D in middle with RHS 138B outer. Length: 4 cm. Width: 1 cm. Shape: Lanceolate. Surface texture: Upper Surface: Smooth. Lower Surface: Downy. Number: There are 3 normally appendaged sepals, and 2 unappendaged sepals with hairy edges. Receptacles: Color: green RHS 144A. Shape: Funnel. Size: Medium, about 1.2 cm×1.2 cm. Surface: Smooth. Peduncle: Length: short, averaging about 5 cm. Surface: Smooth. Color: light green RHS 144A. Strength: Strong.
   Blooms.—Size: Large, average open diameter is about 10 cm. Borne: Singly in clusters of 4-14 blooms. Stems: Strength: Strong, average length is about 80 cm. Diameter: 5 mm. Form: When first open, bloom is cupped. Permanence of bloom: flattens.
   Petalage.—Number of petals under normal conditions: 65. Color: The upper sides of the petals are white/near white RHS 67A. The reverse sides of the petals are RHS N66A, and the based of the petals is yellow-orange RHS 14A. Variegations: N/A. Discoloration: N/A. Fragrance: Slight. Character of fragrance: pure rose.
   Petals.—Texture: Velvety. Size: Width: 4.3 cm. Length: 5.1 cm. Surface: Rough. Shape: Deltoid. Margin: Entire but slightly frilly edge. Apex Shape: Rounded but undulating. Base Shape: Obtuse. Form: Tips slightly recurved. Arrangement: Imbricated. Petaloids: Number: 3. Color: RHS N66A. Length: Anther: 5 mm×1.5 mm, Filament: 5 mm×1 mm, Petal: 25 mm×10 mm. Shape: Half-moon. Margin: Irregular. Persistence: Petals hang on and dry. Lastingness: On the plant: About 8-10 days. As a cut flower: About 8-10 days.
   Reproductive parts.—Stamens: Number: 250. Length: 6 mm. Anthers: Length: 3 mm. Color: RHS 21A. Arrangement: regular around styles. Filaments: Color: Yellow RHS 21A. Length: 3 mm. Pollen: Color: gold yellow RHS 21A. Pistils: Number: 70. Length: 6 mm. Styles: Color: Pale pink RHS 48D. Length: 5 mm (medium). Stigmas: Color: Clear yellow RHS 12A. Length: 1 mm. Hips: None Observed.
Plant characteristics:
   Plant form.—Shrub.
   Plant growth.—Vigorous.
   Age to maturity.—One year.
   Mature plant.—Height: 1.3 m. Width: 0.75 m.
   Rootstock.—Rosa cv. ‘Inermis’.
   Foliage.—Number: Leaflets on normal mid-stem leaves is: 12. Size: Medium, about 16 cm long×12 cm wide. Quantity: Normal. Number of leaves per flowering stem is: 12. Color: New foliage: Upper side: light green RHS 144A with hints of red RHS 178A. Lower side: green RHS 144B. Old foliage: Upper side: dark green RHS 136A. Lower side: green RHS 137B.
   Leaflets.—Size: About 16 cm long×12 cm wide. Shape: Broadly oval. Base Shape: Rounded. Apex Shape: Acuminate. Texture: Leathery. Edge: Serrated. Serrations: Single. Petiole: Color: Green RHS 144A with greyed red RHS 178A. Length: 3.5 cm. Width: 2 mm. Surface Texture: Glandular. Petiole Rachis: Color: green RHS 144A with red RHS 178A on upperside. Underside: Smooth. Stipules: Length: 2.2 cm, smooth and glandular. Color: RHS 144A. Auricle: Shape: Sword-shaped. Length: 6 mm. Width: 0.75 mm. Color: RHS 144A. Vein Color: Same as leaf, RHS 135A with RHS 178A. Venation Pattern: Net-veined (reticulate).
   Wood.—New Wood: Color: Brown-red RHS 178A. Bark: Smooth. Old Wood: Color: Dark-green RHS 136A. Bark: Smooth.
   Branching habit.—Number of Lateral Branches: Few. Lateral Branch Dimensions: Length: 50 cm. Diameter: 6 mm.
   Stems.—Mature Stem: Length: 130 cm. Diameter: 7 mm. Internode Distance: Average. The above measurements are all variable, depending on growing conditions in a season. Stem Pubescence Present: No.
   Prickles.—Quantity: On main canes from base: Ordinary. Number per stem length: 13 per 15 cm. On laterals from main canes: Ordinary. Number per stem length: 13 per 15 cm. Form: Deep concave. Length: 5-6 mm×3-4 mm. Color when young: Red RHS 178B. Color when mature: Brown RHS 166C.
   Small prickles.—Quantity: Main stalk: Few Number per stem length: N/A. Laterals: Few Number per stem length: N/A.
   Disease resistance.—Mildew: Resistant. Blackspot: N/A. Rust: N/A.
   Pest resistance.—Not tested.
   Winter hardiness.—Not tested.
   Growing conditions.—Normal.

What is claimed is:

1. A new and distinct variety of cut rose plant suitable for luxury cut flower production, substantially as herein shown and described.
